(19) United States
(12) Patent Application Publication
(10) Pub. No.: US 2017/0016913 A1
(43) Pub. Date: Jan. 19, 2017

(54) METHODS AND SYSTEM FOR DETERMINING THE DISEASE STATUS OF A SUBJECT

(71) Applicant: OXFORD UNIVERSITY INNOVATION LIMITED, Oxford (GB)

(72) Inventors: Andrew Price, Oxford (GB); Peter Taylor, Oxford (GB); Philippa Hulley, Oxford (GB); Sarah Snelling, Oxford (GB); Chethan Jayadev, Oxford (GB)

(73) Assignee: OXFORD UNIVERSITY INNOVATION LIMITED, Oxford (GB)

(21) Appl. No.: 15/125,781

(22) PCT Filed: Mar. 12, 2015
(86) PCT No.: PCT/GB2015/050732

§ 371 Date: Sep. 13, 2016

(30) Foreign Application Priority Data
Mar. 13, 2014 (GB) 1404518.1
Mar. 14, 2014 (GB) 1404634.6

(51) Int. Cl. G01N 33/68

(52) U.S. Cl. CPC G01N 33/6887; G01N 2800/105; G01N 2800/56; G01N 2800/50; G01N 2800/60; G01N 2800/102

(57) ABSTRACT

A method of determining the osteoarthritis, inflammatory arthritis or joint injury status in a subject, and a panel of test biomarkers for use in determining same is disclosed. In particular, the method comprise the steps of determining the expression levels of at least three test biomarkers in a sample of bodily fluid obtained from the subject; conducting a statistical analysis of the correlation and relative expression levels between the at least three biomarkers; calculating a statistical score based on the statistical analysis; and comparing the statistical score with reference statistical scores generated from at least three reference group expression profiles to predict, diagnose, monitor or determine one or more of osteoarthritis, inflammatory arthritis or joint injury. For both the method and panel the test biomarkers typically contains at least PIIANP. By analysis of the test biomarkers, the disease status of a subject may be determined.

The present invention relates to a novel panel of biomarkers that are able to distinguish between osteoarthritis, inflammatory arthritis and joint injury, and in particular to a panel of synovial fluid protein biomarkers. The invention further provides a method of using the biomarkers to allow differential diagnosis between a number of clinical states, including: (1) osteoarthritis, in particular end stage knee osteoarthritis; (2) joint injury, in particular knee injury; and (3) inflammatory arthritis, in particular inflammatory arthritis of the knee. The ability of the invention to biologically diagnose end-stage knee OA may allow its use as a surrogate endpoint or “virtual knee replacement” in clinical trials for disease-modifying treatments, and in clinical practice to monitor disease progression.

Osteoarthritis is a complex degenerative disorder of the entire synovial joint characterised by progressive loss of articular cartilage, subchondral bone remodelling and variable degrees of synovial inflammation. Patients present with joint pain, swelling and stiffness, loss of mobility and function, and a reduced quality of life. Symptoms can be assessed by clinical observation and patient history and/or by using imaging techniques such as X-ray and MRI.

End-stage osteoarthritis is defined as clinically severe and structurally advanced disease, refractory to non-operative management, suitable for joint replacement surgery. This usually involves significant cartilage loss with areas of bone-on-bone contact. In the context of the knee, joint replacement may be a total or partial replacement, or arthroplasty. During this procedure the damaged joint or joint compartment is removed and replaced with a plastic or metal device. Side effects of this surgery include infections at the incision site and blood clots. Recovery from this procedure takes several weeks and requires extensive physical and occupational therapy.

Joint injury may be defined as the bruising, straining, tearing or rupture of the ligaments or/and tendons, or/and damage to the semi-lunate meniscal cartilages and/or traumatic damage to the articular cartilage. The severity of the injury sustained has a direct impact on the time needed to rehabilitate the joint and recover full function. For mild ligament or tendon injuries, days to weeks of rest and minimal use of the injured joint are needed to achieve full healing, while for moderate or severe injuries, complete healing may require months to years, with accompanying reconstructive surgery to reattach or repair damaged ligaments, tendons or cartilage. In particular, joint injury may refer to cruciate ligament or meniscal injury of the knee joint, which may require surgery, without initial evidence of degenerative changes or articular cartilage injury (as confirmed by MRI and at surgery). There is strong evidence that such injuries increase the risk of future development of osteoarthritis, irrespective of treatment.

Inflammatory arthritis refers to a number of related musculoskeletal conditions where joint (e.g. the knee) damage occurs as a result of an inflammatory process. Common examples are rheumatoid or psoriatic arthritis.

There are presently no reliable, quantifiable and easily measured biomarkers that enable diagnosis, prognosis or monitoring of effect at the individual level for osteoarthritis. As osteoarthritis is a heterogeneous disease of the entire synovial joint with a complex myriad of pathological processes, it is not entirely surprising that the pursuit of single biomarkers has thus far proved largely unsatisfactory. A multi-marker approach comprising a profile of several combined biomarkers may be more appropriate.

Synovial fluid bathes the articular space and acts as a medium of communication for the triangular relationship between synovium, cartilage and bone that is central to the pathophysiology of osteoarthritis. The identification of a signature or “fingerprint” for end-stage knee osteoarthritis from synovial fluid proteins would be valuable to monitor disease progression and assess interventions.

It is an objective of the present invention to diagnose osteoarthritis through a novel synovial fluid biomarker panel. The nature of the invention additionally provides a means to distinguish between one or more of osteoarthritis, joint injury and inflammatory arthritis.

According to a first aspect of the present invention, there is provided a method of determining the osteoarthritis, inflammatory arthritis or joint injury status in a subject, the method comprising: determining the expression levels of at least three test biomarkers in a sample of bodily fluid obtained from the subject; conducting a statistical analysis of the correlation and relative expression levels between the at least three biomarkers; calculating a statistical score based on the statistical analysis and comparing the statistical score generated from the expression levels of the test biomarkers in the test sample with reference statistical scores generated from at least three reference group expression profiles to predict, diagnose, monitor or determine one or more of osteoarthritis, inflammatory arthritis or joint injury wherein the test biomarkers contains at least PIIANP.

The phrase “osteoarthritis status”, “inflammatory arthritis status” or “joint injury status” includes any distinguishable manifestation of osteoarthritis, inflammatory arthritis or joint injury. In particular the method of the invention allows osteoarthritis, and in particular end stage osteoarthritis, inflammatory arthritis or joint injury to be distinguished from each other. In a preferred embodiment the method relates to the knee and to allow the diagnosis of osteoarthritis, and in particular the diagnosis of end stage osteoarthritis, such as end stage knee osteoarthritis. The method of the invention may allow distinguishing between end stage knee osteoarthritis and inflammatory arthritis of the knee and/or knee injury.

It may be possible to use the method of invention to identify patients at much earlier stages of osteoarthritis disease and to monitor disease progression or the effectiveness or response of a subject to a particular treatment. For example, a patient may be diagnosed with osteoarthritis, either by the method of the invention or by other clinical parameters, a therapy for osteoarthritis may then be administered to the patient, and by analyzing a sample from a patient after treatment the efficacy of the administered therapy can be assessed.

As noted above, pursuit of individual biomarkers to indicate the presence or otherwise of osteoarthritis has proved to be difficult. The first aspect of the present invention provides an alternative approach that instead analyses a sample for the expression levels of three or more test biomarkers. By comparing the relative expression levels and internal correlation structure to at least three reference expression profiles rather than to the absolute expression levels of each respective biomarker, the statistical relevance of the profile of the expression levels can be analysed to provide a statistical score that is indicative of the osteoarthritis (or not) or other conditions. It can then be determined if the test sample likely came from a subject suffering from or likely to suffer from end stage osteoarthritis or one of the other conditions.

Preferably, the method does not include the step of obtaining the sample of bodily fluid from a subject.

Preferably, each of the at least three reference group expression profiles define reference expression levels of the at least three test biomarkers obtained from one or more reference samples.

According to a second aspect of the present invention, there is provided a panel of test biomarkers for use in determining the osteoarthritis status, inflammatory arthritis status or joint injury status of a subject, or for predicting, diagnosing, monitoring, or determining osteoarthritis, inflammatory arthritis or joint injury, in a subject; the panel comprising at least two of the following: i) a biomarker associated with inflammatory disease, such as rheumatoid arthritis; ii) a biomarker associated with osteoarthritis; and iii) a biomarker associated with joint injury wherein the test biomarkers contains at least PIIANP.

The expression levels of the test biomarkers in the panel may be determined in 2 or more individuals, preferably more than 10, 20, 30, 40 or 50 individuals with a pre-existing and pre-diagnosed condition, the levels observed may then be statistically analysed to provide a reference group expression profile for different conditions, for example for osteoarthritis, inflammatory arthritis or joint injury.

By providing a panel/profile of test biomarkers, a bodily fluid may be easily tested to determine the presence or (absolute) levels of expression of the biomarkers. By providing at least three biomarkers wherein a positive absolute expression level of each biomarker can be associated with at least one of osteoarthritis, inflammatory arthritis or joint injury, the expression levels may then be statistically analysed according to the method of the first aspect to determine an accurate diagnosis of osteoarthritis, inflammatory arthritis or joint injury.

The bodily fluid sample may be blood, plasma, serum, urine, spinal fluid or synovial fluid. Preferably, the bodily fluid is synovial fluid.

In preferred embodiments, the test biomarkers or biomarker panel or biomarker profile may comprise at least 3, at least 4, and preferably at least 5 biomarkers; and more preferably at least 8 biomarkers. Providing a larger suite of biomarkers increases the accuracy of the predicting, diagnosis, monitoring or determination. However, it has been found that statistically significant results may be obtained by fewer biomarkers.

Whilst the absolute level of expression of at least one biomarker of the test biomarkers may be indicative of inflammatory disease, such as rheumatoid arthritis and at least one biomarker of the test biomarkers may be indicative of osteoarthritis, the absolute expressions by themselves are not conclusive in allowing an accurate diagnosis to be made. Part of the reason, for example, is that the biomarkers used to suggest osteoarthritis may also be expressed in inflammatory arthritis, injury, etc. As such, a positive absolute expression of a single biomarker by itself cannot definitively diagnose, monitor or determine the presence or state of osteoarthritis. However, by providing at least one test biomarker suggestive of end stage osteoarthritis together with at least one test biomarker suggestive of inflammation, statistical analysis can be used to distinguish between samples from individuals with osteoarthritis, inflammatory arthritis or joint injury providing you have a reference profile for the test biomarkers from subjects with each of osteoarthritis, inflammatory arthritis and joint injury. Additionally, by providing such a suite of test biomarkers, the statistical analysis allows distinguishing between end stage osteoarthritis and injury.

Preferably, the test biomarkers, the biomarker panel or the biomarker profile contains at least PIIANP. This biomarker may provide a high statistical weighting to assist in distinguishing the expression levels of two or more test biomarkers in patients suffering from osteoarthritis, in particular end stage osteoarthritis, from the expression levels of two or more test biomarkers in patients suffering from other conditions such as rheumatoid arthritis or injury. However, as described above, it is also possibly indicative of inflammation when considered alone and therefore is not definitive in diagnosing osteoarthritis by itself.

The test biomarkers, the biomarker panel or the biomarker profile may comprise at least 3, 4, 5, 6, 7, 8, 9, 10 or more proteins or fragments thereof. Preferably there are at least 5, more preferably at least 8 or more proteins or fragments thereof.

The test biomarkers, the biomarker panel or the biomarker profile may comprise at least TIMP-1, ADAMTS-4 and PIIANP or fragments thereof,

The test biomarkers, the biomarker panel or the biomarker profile may comprise one or more, two or more, three or more, four or more, or all of IL-6, MCP-1, IP-10, TGF-β3, and COMP or fragments thereof,

The test biomarkers, the biomarker panel or the biomarker profile may comprise one or more, two or more, three or more, four or more, the five or more, six or more, seven or more, eight or more, nine or more, ten or more, of TNF-α, IL-6, IL-8, IL-12, IL-15, MCP-1, IP-10, Eotaxin, TGF-β1, TGF-β2, TGF-β3, MMP-1, MMP-3, MMP-9, COMP, and DcR3 or fragments thereof.

In an example the test biomarkers, the biomarker panel or the biomarker profile may include at least, or consist of, TNF-α, IL-6, IL-8, IL-12, IL-15, MCP-1, IP-10, Eotaxin, TGF-β1, TGF-β2, TGF-β3, MMP-1, MMP-3, MMP-9, COMP, DcR3, TIMP-1, ADAMTS-4, PIIANP or fragments thereof.

In another example the test biomarkers, the biomarker panel or the biomarker profile may include at least, or consist of, IL-6, MCP-1, IP-10, TGF-β3, ADAMTS-4, TIMP-1, COMP and PIIANP or fragments thereof.

The test biomarkers, the biomarker panel or the biomarker profile may also include MMP-13 and/or IL-1RA, or fragments thereof.

The test biomarkers, the biomarker panel or the biomarker profile may also include one or more, two or more, three or more, four or more, five or more, six or more, seven or more, eight or more, nine or more, ten or more further proteins or fragments thereof.

In a preferred embodiment, the step of comparing the expression levels with at least three reference group expression profiles can further comprise querying a database of reference expression levels of the test biomarkers from a plurality of reference samples, wherein the database of known expression levels comprises the expression levels of at least the test biomarkers for samples of bodily fluids taken from subjects diagnosed with one or more of inflammatory arthritis, injury and osteoarthritis, in particular end stage osteoarthritis. Preferably the database comprises samples from at least 10 subjects with each condition. Preferably the data base is queried by undertaking statistical analysis, such as a multivariate statistical analysis, preferably a least squares fit analysis such as a partial least squares regression analysis and more preferably a partial least squares discriminant analysis, of the known reference expression levels against the expression levels of the test biomarkers in the sample from the subject being tested/studied.

In further complimentary and preferred embodiments, at least one reference group expression profile may be obtained by: analyzing reference samples obtained from patients with a known diagnosis of either injury, inflammatory arthritis or osteoarthritis; measuring the reference samples for the reference expression levels of at least the test biomarkers; undertaking statistical analysis of the reference expression levels of each reference sample relative to the reference expression levels of the ensemble of reference samples to determine a relative expression profile for each reference sample; and generating a reference group expression profile by mapping the relative expression profile of each reference sample to the known diagnosis of each reference sample.

Additionally, the step of calculating a statistical score may further comprise: undertaking statistical analysis of the expression levels of the test biomarkers in the test sample relative to the expression levels of the ensemble of reference samples to determine a relative test expression profile for the test sample; and determining the statistical fit between the relative test expression profile and the relative expression profile and assigning a statistical score based on the statistical fit.

By referencing the obtained expression levels with a database of reference samples of known reference expression levels and known diagnoses, statistically relevant patterns may be drawn between the obtained expression levels and the reference expression levels. In essence, each newly obtained series of expression levels of the test biomarkers is compared to the reference expression levels for the test biomarkers and mapped onto the statistical distribution obtained between the reference expression levels and profiles and the diagnosis associated with each reference expression level and profile. This allows a statistical score to be attributed to the obtained test sample relative to the reference samples or diagnoses used to generate the reference expression levels and profiles. For example, a higher score may represent an increased likelihood that the test sample is from a subject suffering from end stage osteoarthritis. The score may be a value that indicates how closely the test sample conforms to the expression profile of the determined diagnosis.

The statistical analysis may use partial least squares fit techniques and optionally or preferably may use partial least squares discriminant analysis. The statistical analysis may be a multivariate statistical analysis. The statistical analysis may interrogate the internal correlation structure and relative expression levels between the at least three biomarkers However, other statistical techniques may be used relevant to the regression analysis performed. It can be appreciated that different statistical techniques may provide differing confidence intervals. The choice of statistical technique is typically dependent upon the suitability of the data.

According to a third aspect of the present invention, there is provided a method of determining the osteoarthritis, inflammatory arthritis or joint injury status in a subject, the method comprising: determining the expression levels of at least three test biomarkers in a sample of bodily fluid obtained from the subject; conducting a statistical analysis of the correlation and relative expression levels between the at least three biomarkers; calculating a statistical score based on the statistical analysis and comparing the statistical score generated from the expression levels of the test biomarkers in the test sample with reference statistical scores generated from at least three reference group expression profiles to predict, diagnose, monitor or determine one or more of osteoarthritis, inflammatory arthritis or joint injury wherein the test biomarkers.

According to a third aspect of the present invention, there is provided a panel of test biomarkers for use in determining the osteoarthritis status, inflammatory arthritis status or joint injury status of a subject or for predicting, diagnosing, monitoring, or determining osteoarthritis, inflammatory arthritis or joint injury in a subject, the panel comprising at least two of the following test biomarkers: i) a biomarker associated with inflammatory disease, such as rheumatoid arthritis; ii) a biomarker associated with osteoarthritis; iii) a biomarker associated with joint injury.

For the panel aspect, the expression levels of the test biomarkers may be used to perform the method of the first or third aspect.

According to a fifth aspect of the present invention, there is provided a system for calculating the probability that a subject has osteoarthritis, inflammatory arthritis or an injury, the system comprising: a test sample of bodily fluid obtained from a subject; a panel of test biomarkers; a processor for undertaking statistical analysis on the expression levels of the biomarkers from the panel; a database containing one or more reference group expression profiles; and a output device for signalling the results of the statistical analysis, wherein the processor determines a statistical score based on a comparison between the expression levels of the panel of test biomarkers in the test sample and the reference group expression profiles representing the probability that the expression levels of the test biomarkers in the test sample diagnose osteoarthritis, inflammatory arthritis or an injury, and in particular end stage osteoarthritis.

The system may be used to assist in the diagnosis, prediction, monitoring or determining of end stage osteoarthritis and may also form part of a test available to medical practitioners to assist diagnosis and monitor disease progression.

According to a sixth aspect of the present invention, there is provided a system for undertaking the method of the first or third aspect of the present invention, the system comprising: a panel according to the second aspect of the present invention; a database containing one or more reference group expression profiles; and an output device for displaying the statistical score.

A method of the invention may be used to determine whether an individual has i) osteoarthritis, and in particular end stage osteoarthritis; ii) a joint injury; or iii) inflammatory arthritis. Preferably the method of the invention may be used to determine whether an individual has i) osteoarthritis in the knee, and in particular end stage osteoarthritis; ii) a knee injury; or iii) inflammatory arthritis in the knee.

A method of the invention may also be used to determine the progression of osteoarthritis or an injury, or to monitor the response of osteoarthritis or an injury to a particular treatment or preventive regime. This involves taking sequential samples over time and analysing the levels and pattern of test biomarker expression to see if any changes in disease status are occurring.

Effective drug treatments for OA are being actively sought. However, there are already effective surgical therapies which prolong the life of the joint and either delay or prevent the need for a total joint replacement. High tibial osteotomy realigns the joint to remove pressure from a region that is becoming damaged and this removes pain and slows the progression of cartilage loss in the joint for several years. The recovery or partial recovery of the joint should be reflected in the synovial fluid fingerprint which will shift from end-stage OA towards injury or the as yet undefinable “normal”. Similarly, the medial or inner side of the knee is more frequently affected by OA than the lateral or outer side. Patients present with undamaged lateral compartments but have a swollen and painful joint due to medial damage. Replacing the medial half of the knee only with a partial or unicopartmental implant typically stabilises the whole joint and removes pain. Evidence suggests that in many cases this prevents further degeneration in the retained articular cartilage. The end stage OA fingerprint should shift back towards “injury or “normal” and remain there for many years. This invention will allow a new way of rapidly and accurately monitoring patients to assess those who have not responded well to surgery. In particular it will be useful to assess when osteotomy patients begin to degenerate again and to assess when they are ready for partial or total knee replacement. Some patients progress much more rapidly than others and a tool which could quantify this rate of progression would be valuable in managing resources, because rapid progressors could be accelerated for surgery, while slow progressors could be managed conservatively. Critically the invention would allow identification of this subgroup of patients at an earlier stage of their rapid progression than is possible at present. Similarly, patients with an early history of joint injury are at accelerated risk of early OA because of either initiated damage or induced instability of the joint when the supporting ligaments fail to heal perfectly (e.g. instability secondary to chronic anterior cruciate ligament reconstruction). These patients could be monitored for rapid or slow progression of joint damage, allowing rational clinical decision making on when or if they should undergo osteotomy or joint replacement. Pain scores and imaging modalities correlate very poorly with actual structural damage, which is irreversible. Therefore patients without high pain go untreated and are subjected to potentially avoidable further joint damage, when early detection could have prioritised them for an osteotomy or partial replacement, thus saving the rest of their knee. Importantly, as new biological or drug therapies are developed an accurate quantitative tool that can measure response to treatment is lacking for OA and would be provided by this invention. Clumsy tools like pain scores and low definition imaging will make interpreting trial data, where structural disease modification is being assessed, approximate at best.

The method of the invention may also be used to monitor osteoarthritis or joint injury progression, and/or to monitor the efficacy of treatments/preventive regimes administered to a subject. This may be achieved by analyzing samples taken from a subject at various time points following initial diagnosis and monitoring the changes in the biomarker panel expression profile. In this case reference levels may include the initial levels/expression profile of the biomarkers in the subject, or the levels/expression profile of the biomarkers in the subject when they were last tested, or both.

In a further example relevant to the present invention the method of the invention may be used to determining the appropriate treatment for a subject.

By way of example, if the analysis of the biomarker profile indicates knee injury, you may advise physiotherapy and/or weight loss to prevent further damage and to avoid the injury developing into osteoarthritis. Alternatively, if the analysis of the biomarker profile indicates a shift towards end stage osteoarthritis, you may advise treatment with a novel therapy or high tibial osteotomy or (partial or total) joint replacement, or you may try physiotherapy but with a means to monitor efficacy of the treatment by looking for a move towards a profile typical of knee injury rather than osteoarthritis.

In a further aspect, the invention may provide the use of a panel test biomarkers or expression profile of test biomarkers according to any other aspect of the invention for determining the osteoarthritis, inflammatory arthritis or joint injury status of a subject.

The invention may further provide use of the determination of the expression profile of a biomarker panel of the invention in a synovial fluid sample for identifying the osteoarthritis, inflammatory arthritis or knee injury status of a subject, in particular for identifying end stage osteoarthritis in a subjects knee.

According to another aspect of the invention there is provided a method of choosing the most appropriate treatment for a subject with joint injury or pain by performing the method of the invention on a sample, preferably a knee synovial fluid sample, from the subject and administering treatment based on the observed levels/profile of test biomarkers in the sample.

According to a still further example relevant to the present invention there is provided a diagnostic reagent for osteoarthritis comprising antibodies for test biomarkers in a biomarker panel of the invention. The reagents may be provided in a kit. The antibodies may be on a chip for high throughput screening. A kit could comprise a multi-well plate or microfluidic card or multi-plex chip prepared with reagents to capture and quantify the markers constituting the biomarker panel or fingerprint, as well as a database containing disease reference profiles and a computer module facilitating comparison of the test results with the reference panel using appropriate statistics. Equipment needed to read the plate or microfluidic card or chip would be standard high throughput laboratory equipment such as Luminex or Mesoscale Discovery or quantitative PCR or microarray platforms.

The kit may comprise instructions for suitable operational parameters in the form of a label or separate insert. The instructions may inform a consumer about how to collect the sample.

The level of a test biomarker present in a sample may be the concentration of the biomarker protein in the sample.

The level of one or more of the biomarkers discussed herein in a sample from said subject may be determined by any suitable assay, which may comprise the use of any of the group comprising immunoassays, mass spectrometry, western blot, ELISA, immunoprecipitation, slot or dot blot assay, isoelectric focussing, SDS-PAGE and antibody microarray immunohistological staining, radio immuno assay (RIA), fluoroimmunoassay, an immunoassay using an avidin-biotin or streptoavidin-biotin system, quantitative PCR etc and combinations thereof.

The level of one or more biomarkers may be determined using targeted tandem mass spectrometry (MS) methods. Examples of such methods include the: accurate inclusion mass spectrometry (AIMS), and quantitative selection reaction monitoring (Q-SRM).

The method of the invention may be carried out in vitro.

The subject may be a mammal and is preferably a human, but may alternatively be a monkey, ape, cat, dog, cow, horse, rabbit or rodent.

Preferably the subject or animal is a human.

The skilled man will appreciate that preferred features of any one embodiment and/or aspect of the invention may be applied to all other embodiments and/or aspects of the invention.

The present invention will be further described in more detail, by way of example only, with reference to the following figures in which:

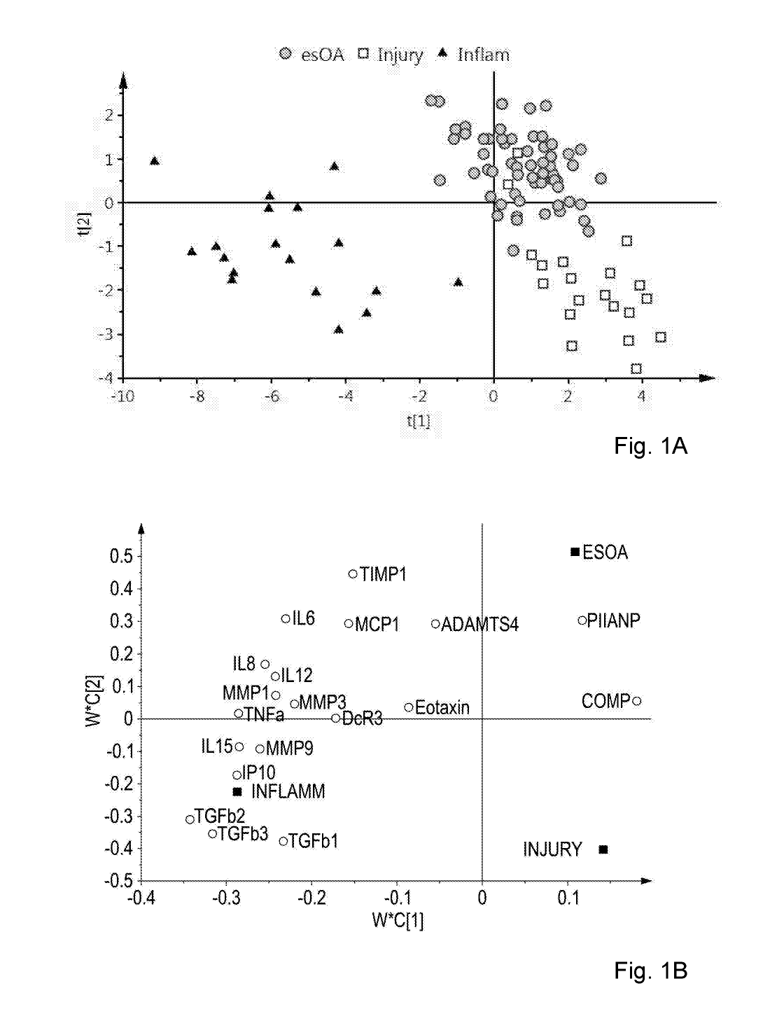

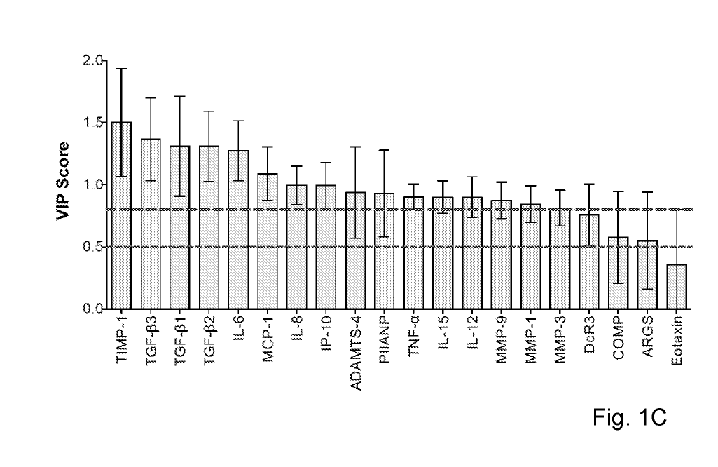

FIG. 1—shows a PLS-DA model using 20 quantitative synovial fluid analytes. FIG. 1A is an Observation Score Plot. FIG. 1B is a Variable Loading Bi-plot. FIG. 1C shoes Variable Importance for Projection (VIP) Scores. Three-component model: R2=0.765; Q2=0.710. End-stage knee osteoarthritis (esOA); knee injury (Injury); inflammatory arthritis (Inflam).

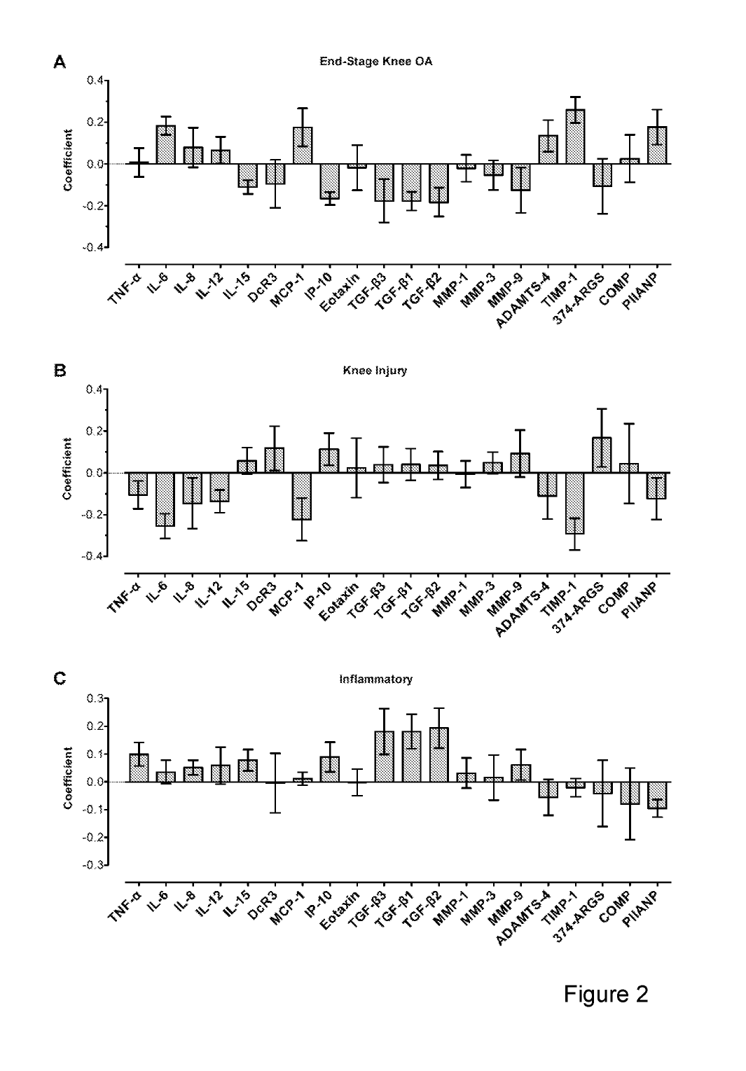

FIG. 2—shows Variable coefficient plots for End-stage knee osteoarthritis (FIG. 2A), Knee Injury (FIG. 2B) and Inflammatory patients (FIG. 2C). PLS-DA model coefficients for 20 quantitative markers (3-components; R2=0.765; Q2=0.710). 95% confidence intervals for regression coefficients are shown and non-significant markers are given in clear columns.

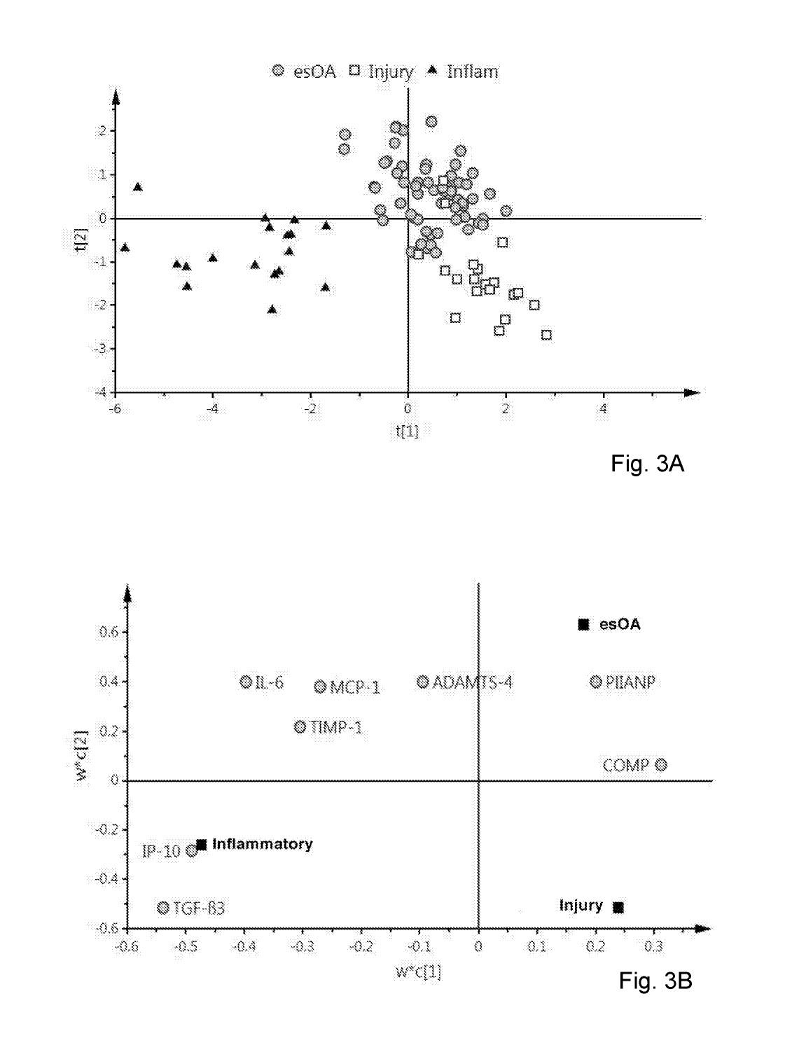

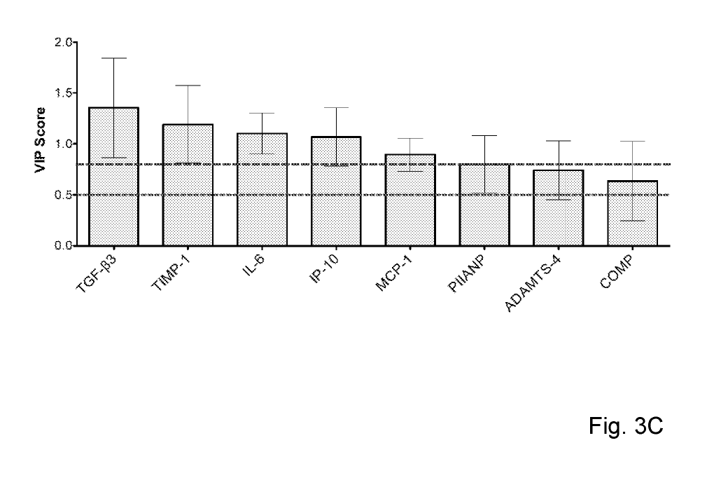

FIG. 3—shows the results of a streamlined PLS-DA model using 8 quantitative synovial fluid analytes. FIG. 3A is an observation Score Plot. FIG. 3B is a Variable Loading Bi-plot. FIG. 3C shows Variable Importance for Projection (VIP) Scores. Two-component model: R2=0.694; Q2=0.673. End-stage knee osteoarthritis (esosteoarthritis); knee injury (Injury); inflammatory arthritis (Inflam).

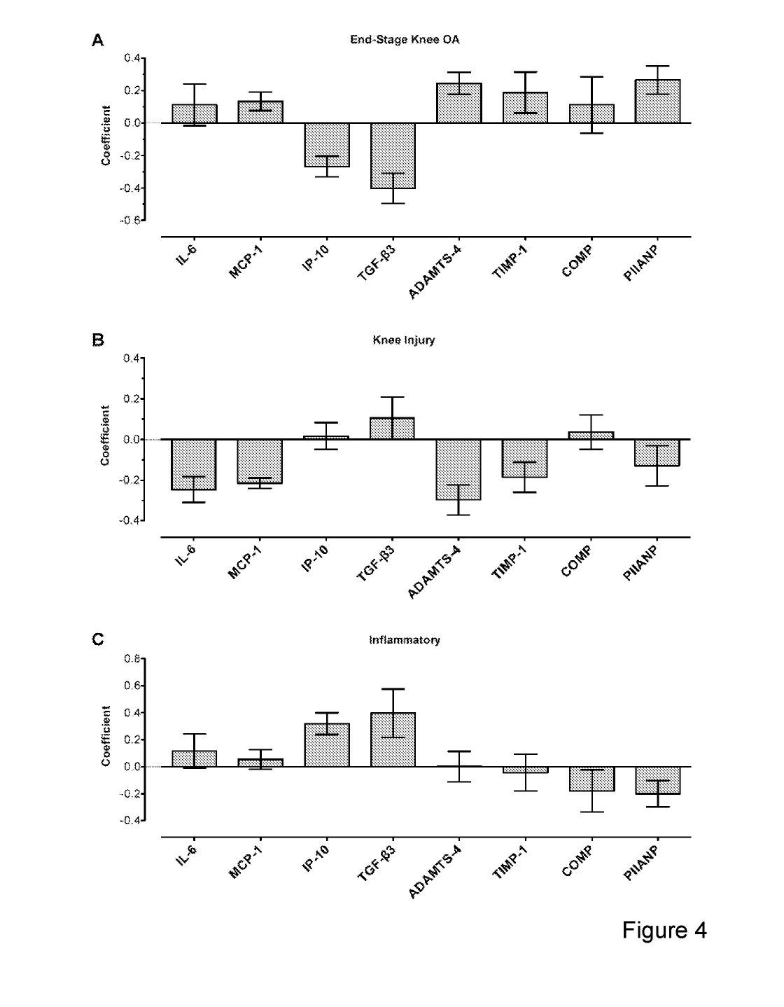

FIG. 4—shows variable coefficient plots for End-stage knee osteoarthritis (FIG. 4A), Knee Injury (FIG. 4B) and Inflammatory patients (FIG. 4C). Streamlined PLS-DA model coefficients for 8 quantitative markers (2-components; R2=0.694; Q2=0.673). 95% confidence intervals for regression coefficients are shown and non-significant markers are given in clear columns.

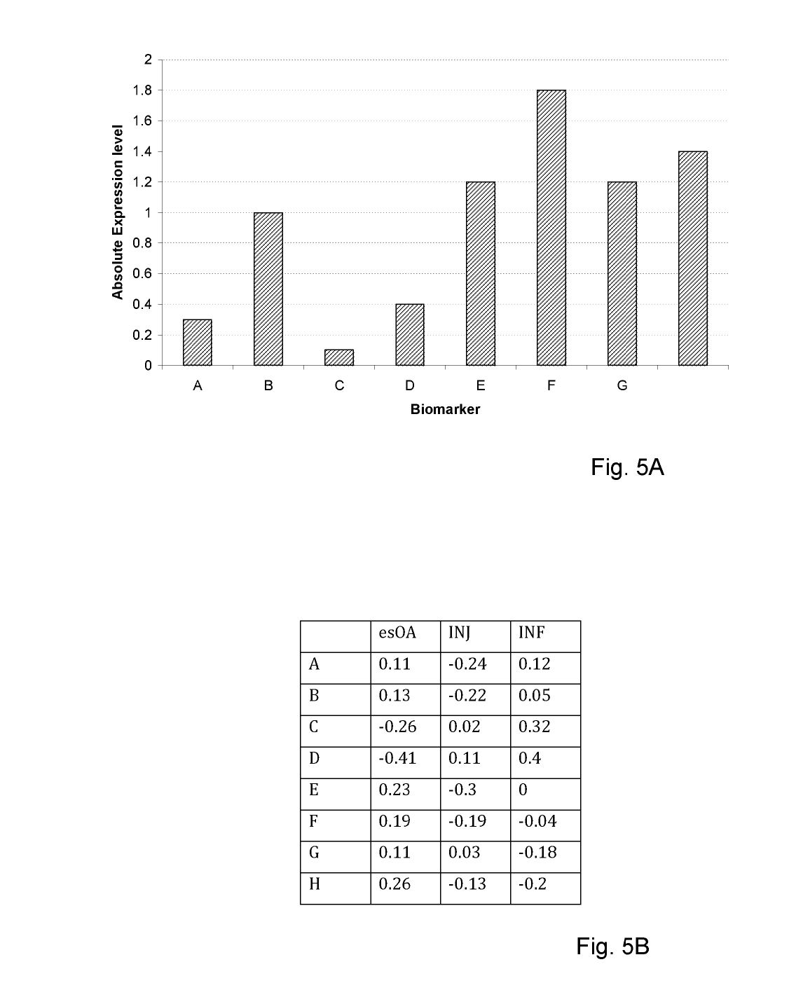

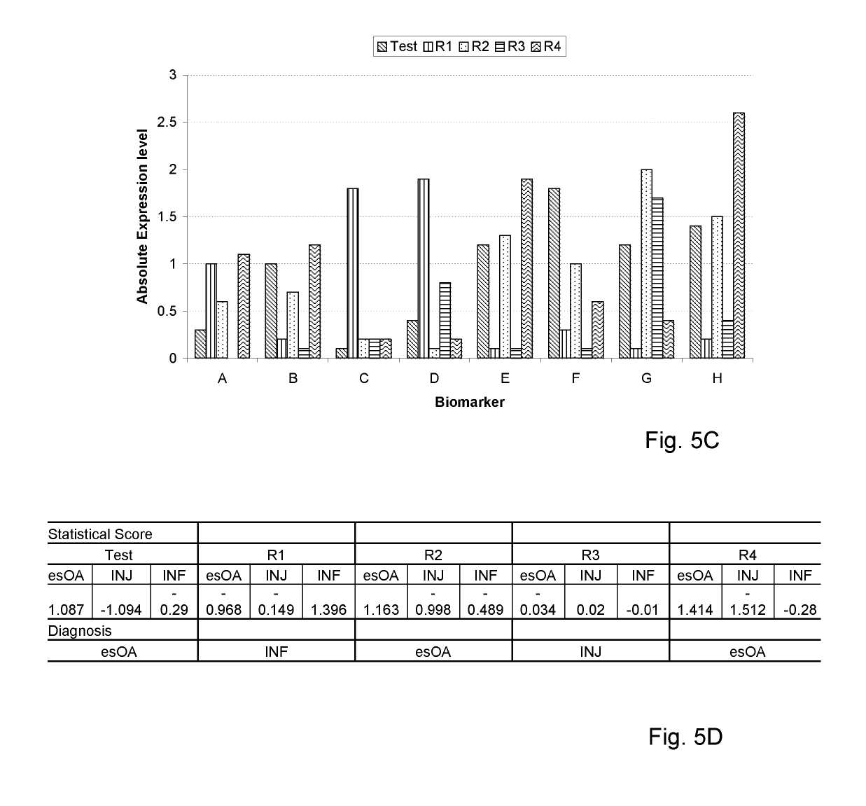

FIG. 5—shows an example of analysis and diagnosis of a test sample according to aspects of the present invention. Absolute expression levels of the test sample for biomarkers A-H are shown in FIG. 5a, the relative coefficients of biomarkers A-H in diagnosing either end stage osteoarthritis (es-OA), Injury (INJ) or Inflammation (INF) are shown in FIG. 5b. FIG. 5c shows the absolute expression levels of test sample of FIG. 5a when compared to the absolute expression levels of biomarkers A-H of reference samples R1-R4. FIG. 5d tabulates the statistical score of the test sample and reference samples R1-R4 when compared to the statistical relative coefficients of FIG. 5b and also then provides an indication of the diagnosis of the test sample.

### MATERIALS & METHODS

Patient Cohorts

End-stage knee osteoarthritis was defined as clinically severe and radiologically advanced disease, refractory to non-operative management, being treated by knee replacement surgery.

Patients with anteromedial OA, with or without patello-femoral OA, and tri-compartmental OA were grouped into a single end-stage knee OA (esOA) cohort. Patients with non-osteoarthritic knee injury (Injury) and inflammatory knee arthritis (Inflammatory) were used as reference groups. Additional SF samples from patients with lateral compartment OA undergoing arthroplasty were prospectively collected and analysed. These patients provided a validation test group for the multivariate models.

Anteromedial gonarthrosis (AMG): Patients with degenerative changes confined to the antero-medial portion of the medial tibio-femoral articulation. The lateral tibio-femoral articular surfaces are well preserved with an intact meniscus. This confined pattern of disease was confirmed radiologically (radiographs and/or MRI) and intra-operatively. Furthermore, patients have intact and functioning anterior and posterior cruciate ligaments, fully correctable varus deformity and little)(<15° to no flexion deformity. Patients had either isolated anteromedial osteoarthritis or anteromedial osteoarthritis plus patello-femoral osteoarthritis.

Lateral compartment osteoarthritis (LcOA) Patients with degenerative changes confined to the lateral tibio-femoral articulation. The central part of the lateral tibial articular surface and the posterior aspect of the femoral condyle are usually involved. The medial tibio-femoral articular surfaces are well preserved. This confined pattern of disease was confirmed radiologically (radiographs and/or MRI) and intra-operatively. Furthermore, flexion deformity is uncommon and valgus deformity is usually fully correctable.

Tri-compartmental osteoarthritis (TCOA): Patients with degenerative changes affecting all three compartments of the knee joint: medial and lateral tibio-femoral, and patella-femoral articular surfaces. This global pattern of disease was confirmed radiographically and intra-operatively. Patients often have anterior cruciate ligament damage, fixed varus or valgus deformity and flexion deformity.

Knee injury (Injury): Patients with anterior cruciate ligament and/or meniscal injuries without any clinical, radiological (radiographic and MRI) or arthroscopic evidence of evidence of articular surface degenerative changes or osteochondral defects. The median interval between injury and surgery was 6.5 months (interquartile range 4-9.75 months).

Inflammatory arthritis: Patients with rheumatoid arthritis (RA) or psoriatic arthritis (PsA) affecting the knee joint. Patients were on a range of anti-inflammatory and/or disease modifying treatments.

TABLE 1

| Cohort | Description | N |
|---|---|---|
| esosteoarthritis | Patients with anteromedial osteoarthritis | 60 |
| | (with or without PFosteoarthritis) | |
| | and tri-compartmental knee | |
| | osteoarthritis undergoing either | |
| | primary knee arthroplasty | |
| Injury | Patients undergoing surgery for | 20 |
| | cruciate or meniscal injuries without | |
| | evidence of degenerative changes | |
| | (confirmed by MRI and at surgery) | |
| Inflammatory | Patients with rheumatoid or psoriatic | 18 |
| | arthritis of the knee, with or without | |
| | disease-modifying treatments | |
| Validation | Patients with lateral compartment knee | 10 |
| | osteoarthritis undergoing either primary | |
| | unicondylar or total knee arthroplasty | |

Synovial Fluid Collection & Preparation

Synovial fluid from patients in the end-stage knee osteoarthritis cohorts was obtained by needle aspiration after superficial dissection, but prior to arthrotomy to avoid contamination with blood. Samples were obtained via the medial knee compartment for patients in the AMG and TCOA cohorts, and lateral compartment for patients with LCOA. For patients in the injury cohort, synovial fluid was needle aspirated from the patella-femoral compartment after routine skin preparation and extremity draping, but prior to any surgical incisions. In all cases, lavage samples were not taken because of potentially variable and uncontrolled dilution that would make comparisons unreliable.

Synovial fluid samples were placed in sterile additive free specimen pots and stored immediately at 4° C. pending processing within 4 hours. Samples were centrifuged at 3000 g for 25 minutes at 4° C. to separate solid debris and cells. The supernatant of each sample was aliquoted into separate 500 μl microfuge tubes, snap frozen in liquid nitrogen and stored at −80° C. until analysis.

Biological Panel

The biological analysis of synovial fluid samples comprised the following panel of 34 markers using combination of Luminex and MSD multiplex immunoassays, and ELISA.

| | |
|---|---|
| Pro-inflammatory | IL-1β, TNF-α, IL-6, IL-8, IL-2, |
| cytokines | IL-12, IL-15, GM-CSF |
| Regulatory cytokines | IL-1Ra, IL-4, IL-10, IL-2R |
| Chemokines | RANTES, MIP-1α, MIP-1β, MCP-1, |
| | IP-10, Eotaxin, MIG |
| Growth Factors | TGF-β1, TGF-β2, TGF-β3, BMP-2, BMP-7 |
| Matrix Enzymes | MMP-1, MMP-3, MMP-9, MMP-13, TIMP-1, |
| | ADAMTS-4 |
| Cartilage Turnover | COMP, PIIANP |
| Others (bone) | LIGHT, DcR3 |

At least 50% of samples in each cohort were required to be above the limit of quantification (LOQ) for analyte measurements to qualify for quantitative analysis.

Sample Preparation

Prior to analysis, synovial fluid aliquots were thawed at room temperature and clarified at 10000 g for 10 minutes. The supernatant was then treated with 2 mg/ml bovine testicular hyaluronidase (type I-S, 618.4 U/mg, Sigma. Hyaluronidase treatment entailed 1:1 volume mixture of synovial fluid with 4 mg/ml HAse, vortexing for 5 seconds and incubation at RT for 1 hr on a shaker. Samples were centrifuged at 1000 g for 5 minutes and the supernatant used for the assay. The end result was 2-fold sample dilution with 2 mg/ml (≈1200 U/ml) hyaluronidase. Since synovial fluid samples were aliquoted into small volumes at the time of collection, there were no freeze-thaw cycles prior to analysis.

Immunoassays

Meso Scale Discovery (MSD) platform, the Luminex platform and magnetic-bead Luminex assays were used where possible.

The synovial fluid samples in this study were analysed for the 34 markers in the biological panel by 11 different multiplex or single-plex assay kits. The same platform, assays kit, reagents, lot numbers and protocols were used for each marker throughout the study to analyse all samples. All commercially sourced immunoassays were conducted according to the manufacturer's protocol. Plates, reagents and wash solutions provided by the manufacturers were used in all cases. Custom MSD assays were performed using optimised in-house protocols following MSD guidelines and MSD recommended reagents. On all platforms, calibrators and blanks were measured in duplicate. Synovial fluid aliquots were run in duplicate for all assays except polystyrene-bead Luminex assays, where they were run in triplicate.

Luminex Assays

Human cytokine magnetic 25-plex panels were purchased from Life Technologies (LHC0009M). These kits were used to measure pro-inflammatory cytokines IL-2, IL-12 (p40/p70), IL-15 and GM-CSF; regulatory cytokines IL-1Ra, IL-10 and IL-2R; and chemokines RANTES, MIP-1α, MIP-1β, MCP-1, IP-10, Eotaxin and MIG. Bio-Plex Pro TGF-β magnetic 3-plex assays were purchased from Bio-Rad (171-W4001M). VersaMap Custom Premixed MMP-13 polystyrene single-plex kits were purchased from R&D Systems.

A BioTek ELx50 microplate washer was used to perform wash steps for Luminex assays: A vacuum filtration manifold was used for the polystyrene bead assays using filter-bottom plates, and the magnetic separation manifold was used for magnetic bead assays using flat-bottom plates. A Luminex xMAP-200 system used to read plates.

Meso Scale Discovery Assays

Commercial kits were purchased directly from MSD. Human Proinflammatory-4 II Ultra-Sensitive kits (K15025C) were used to measure cytokines IL1-β, IL-6, IL-8 and TNF-α. Matrix enzymes MMP-1, MMP-3, MMP-9 and TIMP-1 were assayed using human MMP 3-plex (K15034A) and TIMP-1 mono-plex (K151JFC) kits respectively.

A proto-type 4-plex (N45ZA-1) was created and validated for BMP-2, BMP-7, LIGHT and DcR3 by MSD's prototype plate printing service. Antibody pairs for sourced from R&D systems human DuoSet ELISA kits: BMP-2 (DY355), BMP-7 (DY354), LIGHT/TNFSF14 (DY664) and DcR3/TNFRSF6B (DY142). A series of in-house quality control experiments were conducted before the use in the study to confirm acceptable cross-reactivity, non-specific binding and background signals.

A BioTek ELx50 microplate washer (Oxford) or Molecular Devices SkanWasher 300 (GSK) was used for automated plate washing. A MSD Sector Imager 6000 was used to read plates.

ELISA

Human ADAMTS-4 ELISA kits were purchased form CusaBio (CSB-EL0001311HU). Human COMP ELISA kits were purchased from BioVendor (RD194080200). Human PIIANP ELISA kits were purchased from Millipore (EZPIIANP-53K). A Molecular Devices SkanWasher 300 was used for automated plate washing and a Molecular Devices SpectraMax plate reader was used to read all ELISA plates.

Beta-Substitution for Left Censored Data

This study used the β-substitution method for left-censored data with single or multiple censor points (i.e. LOQ and LOD) described by Ganser & Hewett. The distribution of uncensored data is used to calculate a β factor, which is then used to adjust the limit before substitution. The procedure involves separate substitutions to provide summary statistics for the naive data and natural-log transformed data. A sample size of n>5 is required and data censoring of up to 50% can be handled with performance comparable to the gold standard MLE. For each analyte, the proportion of samples below LOQ and below LOD in each cohort was recorded. Beta-substitution was performed only if fewer than 50% of samples in the cohort were below LOQ. For each analyte with left-censored data, the β-substitution procedure was conducted separately for patient cohorts, which were treated as distinct data arrays. Samples below LOQ (and above LOD) were substituted with β.LOQ and samples below LOD were substituted with β.LOD. Calculations were made in Microsoft Excel using a template with formulae provided in the original article. If more than 50% of samples in the cohort were below LOQ, then the marker was excluded from further analysis as a continuous variable. However, these markers were retained for qualitative categorical analysis.

Partial Least Squares Discriminant Analysis

Synovial fluid analyte concentrations were first natural logarithm transformed to minimise data skew and then (mean) centred and scaled to unit variance, to allow all markers irrespective of range to have equal weight in the analysis. Qualitative markers were assigned as dummy variables coded 0 for <LOQ and 1 for >LOQ.

Supervised partial least squares discriminant analysis (PLS-DA) was used to determine the most parsimonious way to distinguish between end-stage knee osteoarthritis, knee injury and inflammatory arthritis on the basis of synovial fluid measurements. PLS-DA was conducted separately for quantitative and categorical synovial fluid (SF) analytes and was implemented with the NIPALS algorithm. Predictive models were produced with study cohort as the categorical dependent Y-variable and the SF analytes as the explanatory X-variables. The R2 value was used to estimate goodness of model fit i.e. how well the model fits the data. With numerous and correlated X-variables there is a risk for “over-fitting”, i.e. getting a well fitting model with little or no predictive power. Therefore a process of internal cross validation was conducted to generate a Q2 value as an estimate of the model's predictive quality i.e. how well the model predicts new data. A total of 7 rounds of cross validation were conducted and a Q2>0.5 was considered acceptable. Low R2 and/or Q2 values indicate that the relationship between X and Y is poor or there is significant noise in the data. The number of latent projections (components) used in the model was determined by the compromise between optimum R2 and Q2 values i.e. the model was stopped at maximum cumulative Q2 value. Observation score plots were produced to visually assess cohort class separation. Variable loading weights bi-plots were produced to display the relationship between analytes and cohorts. Analytes (X-variables) in the vicinity of a dummy cohort (Y-variable) have the greatest discriminating power.

Model Performance

A confusion matrix was used to assess PLS-DA models by comparing actual cohort to predicted cohort. Model accuracy and reliability was calculated for each study cohort. Accuracy is the number of patients predicted correctly as a percentage of the total number of patients actually in the cohort. This is equivalent to sensitivity. Reliability (or precision) is the number of patients predicted correctly as a percentage of the total number of patients predicted to be in the cohort. This is equivalent to positive predictive value. The average (mean) accuracy and reliability was calculated for each model. The overall accuracy of the model was given by the total number of patients correctly classified (i.e. true positives) as a percentage of the total number of patients. Specificity is the percentage of patients correctly classified as not belonging to a particular cohort. It was calculated for each cohort by dichotomising the confusion matrix for that cohort e.g. to “group A” and “non-group A”.

Synovial Fluid Marker Importance and Biological Fingerprinting

The variable influence on projection (VIP) parameter (with jack-knifed 95% confidence intervals) is a measure of how each much an X-variable (SF analyte) contributes to the overall PLS-DA model. This includes both its importance to class separation (Y-variable) and its importance to modelling the latent structure of X-variables i.e. components. Analytes with a VIP>0.8 were considered important for the overall model; VIP between 0.8 and 0.5 considered potentially important, and VIP<0.5 considered unimportant.

The importance of a given X-variable for Y is proportional to its distance from the origin in the loading space (loading weight bi-plot). These lengths correspond to the PLS regression coefficients, which were therefore used determine how strongly an analyte is associated with a cohort. The coefficient is significant if its (jack-knifed) 95% confidence interval does not include zero. The “biological fingerprint” of each cohort was defined by its combination of analytes with significant PLS regression coefficients. Markers associated with a cohort (i.e. significant positive coefficient) were termed “positive elements” or “ridges” of the fingerprint; those opposing a cohort (i.e. significant negative coefficient) were termed “negative elements” or “troughs”.

Model Streamlining

The PLS-DA process was repeated to obtain a streamlined model with the most parsimonious combination of quantitative SF markers for class discrimination. An iterative approach was used to obtain the greatest R2 and Q2 values with the least number of quantitative markers that all had a VIP>0.5.

Model Validation

The data used to generate the PLS-DA models are known as the “training set”. Identical wide-spectrum SF analysis of new patients was used as “test set” data to validate the PLS-DA models. Ten patients with end-stage (lateral compartment) knee OA that were used as a test cohort. The models are blinded to the cohort membership of these new patients and assessed for their ability to correctly classify them. The predictive performance is assessed as described above.

Data processing and PLS-DA was implemented in SIMCA-P ver. 13.0.2 (Umetrics, Sweden). Coefficient heat maps were created using MeV Ver. 4.8.1 (TG4 Software)

Results

Multivariate Analysis

Multivariate analysis was conducted for 20 quantitative and 12 qualitative synovial fluid analytes. Quantitative markers included 5 inflammatory cytokines (TNF-α, IL-6, IL-8, IL-12 & IL-15); 3 chemokines (MCP-1, IP-10 and Eotaxin), 3 isoforms of TGF-β, 5 matrix enzymes (MMP-1, MMP-3, MMP-9, TIMP-1, ADAMTS-4); 2 markers of cartilage metabolism (COMP & PIIANP); and DcR3. Categorical markers included 2 inflammatory cytokine (IL-1β & GMCSF); 3 regulatory cytokines (IL-1Ra, IL-10 & IL-2R); 4 chemokines (RANTES, MIP-la, MIP-1β & MIG); growth factor BMP-2, matrix enzyme MMP-13 and LIGHT. Three analytes (IL-2, IL-4 & BMP-7) were excluded because they were quantifiable in less than 25% of patients in any group.

Quantitative Synovial Fluid Analytes

PLS-DA using quantitative synovial fluid analytes produced good class separation between the 3 study cohorts (FIG. 1A). A 3-component model was generated that explained 76.5% (R2=0.765) of the variability between patient groups with a predictive quality of 71.0% (Q2=0.710). Only two patients with knee injury were misclassified as having end-stage knee osteoarthritis and all remaining patients were classified correctly.

Ten (of the 10) test patients were classified correctly as end-stage knee osteoarthritis and the remaining patient misclassified as knee injury (accuracy 100%; reliability 100%).

The loading bi-plot suggests PIIANP has a strong discriminatory function for end-stage knee osteoarthritis. TIMP-1, ADAMTS-4, MCP-1 and IL-6 also load towards end-stage knee osteoarthritis. The majority of markers discriminate against knee injury.

All quantitative markers were important (VIP>0.8) for group separation except DcR3 and COMP that were potentially important (0.8>VIP>0.5), and Eotaxin which was not important (VIP<0.5) (FIG. 1C). The marker profile for each cohort according to (significant) model coefficients is shown in (FIG. 2). End-stage knee osteoarthritis was characterized by a marker profile comprising elevated TIMP-1, IL-6, PIIANP, MCP-1, ADAMTS-4 and IL-12 in combination with reduced TGF-β isoforms, IP-10, IL-15 and MMP-9 (FIG. 2A).

Streamlined Model

The streamlined PLS-DA model used 8 quantitative synovial fluid markers: inflammatory cytokine IL-6; chemokines MCP-1 and IP-10; TGF-β3; aggrecanase ADAMTS-4; metalloproteinase inhibitor TIMP-1 and cartilage metabolism markers PIIANP and COMP. Good class separation was achieved with a 2-component model that explained 69.4% (R2=0.694) of the variability between patient groups with a predictive quality of 67.3% (Q2=0.673) (FIG. 3A). The streamlined model had good accuracy and reliability with only 3 knee injury patients being misclassified as end-stage knee osteoarthritis and all other patients were classified correctly.

Ten (of ten) test patients were classified correctly as end-stage knee osteoarthritis and the remaining patient misclassified as knee injury (accuracy 100%; reliability 100%).

The loading bi-plot shows PIIANP discriminates best for end-stage knee osteoarthritis and most markers discriminate against knee injury (FIG. 3B). TIMP-1 and ADAMTS-4 also load favourably towards end-stage knee osteoarthritis. The VIP scores for all 8 markers in the streamlined model were important (TGF-β3, TIMP-1, IL-6, IP-10, MCP-1 & PIIANP) or potentially important (ADAMTS-4 & COMP) (FIG. 3C). The marker profile characterising end-stage knee osteoarthritis comprised elevated PIIANP, TIMP-1, ADAMTS-4 and MCP-1 in combination with reduced IP-10 and TGF-β3.

FIG. 4 shows variable coefficient plots for end-stage knee osteoarthritis, knee injury and inflammatory arthritis demonstrating how a panel of eight biomarkers can be used with multivariate analysis to produce an expression profile which allows the three conditions to be distinguished. The variable expression plots describe the interrelated strengths of biomarkers to suggest or oppose classification of the three conditions.

FIG. 5 shows a hypothetical comparison between a test sample and a database of reference samples. The simple example is intended to illustrate how the invention works and can be used. It is not intended to provide an indication of the expected expression levels, relative or absolute, of the biomarkers, nor is it intended to be representative of the biomarkers chosen for a typical biomarker panel.

In this hypothetical example a bodily fluid test sample is obtained from a subject in a method not covered by the present invention. The test sample is a typically a sample of synovial fluid obtained from the joint, such as the knee, of subject for testing. Generally the subject is suspected of suffering from a joint related condition such as rheumatoid arthritis, osteoarthritis, injury or the like. Crucially, the diagnosis of the test sample is unknown.

Once a sample of synovial fluid is obtained from the subject, the sample is analysed for the concentrations of a series of test biomarkers labelled A, B, C, D, E, F, G and H in FIG. 5. However, it can be appreciated that the number of test biomarkers may be adjusted depending on a large number of factors including desired accuracy of diagnosis and ease of testing.

FIG. 5a shows the expression levels of the test biomarkers (A-H) in a sample 200 in a bar chart format. It may be seen that the absolute expression levels or concentrations when ordered by absolute values are F, H, G, E, B, D, A, C. In the present invention, diagnoses of inflammatory arthritis (INF), end-stage osteoarthritis (es-OA) or injury (INJ) are able to be associated with each sample. Additionally, a relative degree of osteoarthritis severity may also be assigned to relevant samples allowing a sliding scale of osteoarthritis severity to be determined as will be described below.

In the present example, for the test sample, absolute concentration levels (expression levels) of the biomarkers are 0.3 for biomarker A and 1, 0.1, 0.4, 1.2, 1.8, 1.2 and 1.4 for biomarkers B to H respectively. At least on initial comparison of the absolute expression levels of the biomarkers, the diagnosis is unknown.

The reference samples (R1-R4) each have respective absolute concentration levels (expression levels) of the same test biomarkers (A-H). It may be appreciated that additional biomarker absolute concentration levels may also be stored against each reference sample.

The reference samples have been previously subjected to multivariate statistical analysis (project latent structures) of the type described above. As the diagnosis for each reference sample is known, this allows a statistical profile for the set of reference samples reflecting a particular diagnosis to be obtained. The ensemble of statistical profiles may then be analysed to determine relative coefficients of each biomarker in relation to the selected set of biomarkers. Assigning the relative coefficients to each dataset (i.e. each sample) using statistical methods allows a statistical score for the sample to be calculated for each of the possible diagnoses.

In broad terms, the set of biomarker levels are consolidated as a single plot point and plotted against the like plot points of the remaining reference samples, using partial least squares discriminatory analysis. This obtains a graph similar to those shown in FIGS. 1A and 3A and reflects a normalised representation of the relative expression levels of the biomarkers for each sample relative to each other sample in the ensemble. It may be seen from these figures that a clustering of samples associated with a diagnosis is apparent after the multivariate statistical analysis has been performed.

The end result of the reference data analysis is that a series of reference group expression profiles may be obtained characterising each disease according to the expression levels of the biomarkers tested. In other words, a regression or plot can be drawn from the graph of FIGS. 1A and 3A for the effect of the expression levels of each biomarker for each diagnosis. Such reference expression levels are shown in FIGS. 2 and 4 for each of the diagnoses. The reference expression levels describe how each biomarker contributes to the ability to produce a successful diagnosis for that particular set of biomarkers. For example, for the selected group of biomarkers shown in FIG. 4A, the biomarker TGF-β3, has been assigned a negative coefficient for end-stage osteoarthritis, a slightly positive coefficient for knee injury and a strongly positive coefficient for inflammatory disease.

It is important to note that these coefficients represent how strongly or how relevantly each biomarker is correlated to its selected cohorts for each diagnosis. In the example shown in FIG. 4A, TGF-β3 is strongly expressed in the majority of patients with inflammatory disease, such as rheumatoid arthritis. In other words, for a sample from a patient suffering from inflammation, where the selected biomarkers are measured, a high expression level of TGF-β3 strongly supports such diagnosis. However, TGF-β3 is strongly negatively relevant to a diagnosis of end-stage osteoarthritis for this selection of biomarkers. In other words, for this selection of biomarkers, the expression level of TGF-β3 is not helpful by itself for diagnosing end stage osteoarthritis and a high expression level may be negatively associated with a diagnosis of end stage osteoarthritis. Similarly, TGF-β3 is broadly not suggestive or dismissive in diagnosis of injury.

Conversely, PIIANP is positively correlated to a diagnosis of end stage osteoarthritis, for this set of biomarkers, but is negatively correlated to a diagnosis of inflammation or injury. In other words, for this panel or selection of biomarkers, a high expression level of PIIANP is suggestive of end stage osteoarthritis and dismissive of knee injury and inflammation, but not exclusively—only within the ensemble measured.

In particular, this allows the absolute concentration levels of each test sample for a set panel or selection of biomarkers to be analysed to obtain a fit profile to one of the diagnoses expression profiles. In this manner, for an unknown cohort, such as the test sample, the absolute expression levels or concentration of the selection of biomarkers can be statistically analysed against the reference absolute expression levels of the same selection of biomarkers to determine where the absolute expression levels of the test sample lie within the ensemble, i.e. where on the graphs of FIGS. 1A and 3A the test sample lies. The test sample can then be analysed against the expression profiles of each of the cohorts using the biomarker coefficients to plot the test sample data against the expression profiles of each cohort or disease. This allows a statistical score for the test sample against each cohort or disease to be determined. From these scores, the relevant diagnosis can be made by using the statistical score that most closely represents the expression profile for the disease.

It is important to appreciate that it is only by providing biomarkers for more than one cohort or disease that allows the expression profiles obtained from the statistical analysis to be accurate in determining a diagnosis. As may be seen from a comparison of FIGS. 2 and 4, reducing the number of biomarkers influences the statistical coefficients for each biomarker. For example, when a larger number of biomarkers are used, the discriminatory power of IL-6 for diagnosing end stage osteoarthritis is improved. For the 19 biomarkers shown in FIG. 2, IL-6 has a positive coefficient of 0.2 and is statistically significant in discriminating. However, when the number of biomarkers is reduced to 8 as shown in FIG. 4, IL-6's relative significance in comparison to the other test biomarkers is diminished and the coefficient drops to 0.11.

Returning to FIG. 5, FIG. 5b, shows the relative coefficients of the biomarkers A-H for this set or panel of biomarkers. It can be noted that some biomarkers have a positive coefficient and some biomarkers have a negative coefficient for each diagnosis. The relative coefficients may be obtained using the methods described above, namely undertaking multivariate statistical analysis against the absolute expression levels of the biomarkers for the reference samples with known diagnoses to determine the effect or statistical coefficient of each biomarker relative to the selection or panel of biomarkers.

FIG. 5c shows an example of how the absolute expression levels of the biomarkers A-H are expressed in relation to the absolute expression levels of the reference samples R1-R4. It may be noted that patterns are difficult to easily discern, which is particularly evident if the reference sample R1-R4 data is compared to the known diagnosis of each reference sample shown in FIG. 5d. The expression levels from two samples from two different patients suffering from the same condition, such as end stage osteoarthritis may have very different absolute expression levels and even different relative expression level profiles when compared to each other. It is only when the statistical relevance of each biomarker is attributed for that patterns emerge.

FIG. 5d is a table that compares the statistical scores of the test sample with the reference samples for each diagnosis. In the present case, the statistical scores have been obtained by multiplying the expression coefficient of the biomarker with the absolute expression level of each biomarker for each possible diagnosis and each sample. However, it can be appreciated that this is an example only and other statistical methods may be used to obtain a statistical score. Additionally, in the example shown, the diagnosis of each of samples R1-R4 is known. It can be seen that for the test sample, the statistical score for esOA is significantly greater than the statistical score for INJ or INF. A combination of high levels of expression for biomarkers indicative of esOA and a lack of contrary expression for biomarkers counter-indicative results in this high statistical score for this group of biomarkers and this sample. It is also possible to analyse the statistical scores for the other selected diagnoses and the other reference samples. It can be noted that injury is often associated with a lack of other overriding diagnosis.

In the present case, a comparison of the statistical scores suggests that the strongest correlation between the possible diagnoses and the expression levels of the biomarkers in the test sample is end stage osteoarthritis (esOA).

As noted above, additional statistical analysis, such as normalisation of the absolute or relative expression levels may be used to allow a relative comparison to be made between samples to determine their relative position within a scale of severity of the diagnoses. This is particularly the case for end stage osteoarthritis, which may develop from injury and early osteoarthritis into end-stage osteoarthritis. By measuring biomarkers and determining the relative severity of the disease without using invasive techniques, effective treatment regimes relative to the progressive stage of the illness may be administered. This ensures that the correct treatment is provided at the correct time for that patient.

This multivariate statistical approach using a set of biomarkers to positively and negatively discriminate between diseases with similar biomarker responses allows seemingly random distributions of biomarker expression levels to be used to accurately diagnose disease.

1. A method of determining the osteoarthritis, inflammatory arthritis or joint injury status in a subject, the method comprising:
determining the expression levels of at least three test biomarkers in a sample of bodily fluid obtained from the subject;
conducting a statistical analysis of the correlation and relative expression levels between the at least three biomarkers;
calculating a statistical score based on the statistical analysis; and
comparing the statistical score with reference statistical scores generated from at least three reference group expression profiles to predict, diagnose, monitor or determine one or more of osteoarthritis, inflammatory arthritis or joint injury, wherein the test biomarkers contains at least PIIANP.

2. A panel of test biomarkers for use in determining the osteoarthritis status, inflammatory arthritis status or joint injury status of a subject or for predicting, diagnosing, monitoring, or determining osteoarthritis, inflammatory arthritis or joint injury in a subject, the panel comprising at least two of the following test biomarkers:
i) a biomarker associated with inflammatory disease, such as rheumatoid arthritis;
ii) a biomarker associated with osteoarthritis;
iii) a biomarker associated with joint injury,
wherein the test biomarkers contains at least PIIANP.

3. A method of determining the osteoarthritis, inflammatory arthritis or joint injury status in a subject, the method comprising:
determining the expression levels of at least three test biomarkers in a sample of bodily fluid obtained from the subject;
conducting a statistical analysis of the correlation and relative expression levels between the at least three biomarkers;
calculating a statistical score based on the statistical analysis; and
comparing the statistical score with reference statistical scores generated from at least three reference group expression profiles to predict, diagnose, monitor or determine one or more of osteoarthritis, inflammatory arthritis or joint injury.

4. A panel of test biomarkers for use in determining the osteoarthritis status, inflammatory arthritis status or joint injury status of a subject or for predicting, diagnosing, monitoring, or determining osteoarthritis, inflammatory arthritis or joint injury in a subject, the panel comprising at least two of the following test biomarkers:
i) a biomarker associated with inflammatory disease, such as rheumatoid arthritis;
ii) a biomarker associated with osteoarthritis;
iii) a biomarker associated with joint injury.

5. The method of claim 1 or claim 3 or the panel of claim 2 or claim 4, wherein the bodily fluid is synovial fluid.

6. The method of claim 1 or claim 3 or claim 4 or the panel of claim 2 or claim 4 or claim 5, wherein the test biomarkers comprise at least 5 biomarkers; or wherein the test biomarkers comprise at least 8 biomarkers.

7. The method of any preceding method claim, wherein at least one biomarker of the test biomarkers is indicative of inflammation and at least one biomarker of the test biomarkers is indicative of osteoarthritis.

8. The method of any preceding method claim or the panel of any preceding panel claim, wherein the test biomarkers comprise at least 3, 4, 5, 6, 7, 8, 9, 10 or more proteins or fragments thereof, and preferably at least 5, and more preferably at least 8 or more proteins or fragments thereof.

9. The method of any preceding method claim or the panel of any preceding panel claim, wherein the test biomarkers comprise at least IP-10, TIMP-1, ADAMTS-4 and PIIANP or fragments thereof.

10. The method of any preceding method claim or the panel of any preceding panel claim, wherein the test biomarkers comprise one or more, two or more, three or more, four or more, or all of IL-6, MCP-1, IP-10, TGF-β3, and COMP or fragments thereof,

11. The method of any preceding method claim or the panel of any preceding panel claim, wherein the test biomarkers comprise one or more, two or more, three or more, four or more, the five or more, six or more, seven or more, eight or more, nine or more, ten or more, of TNF-α, IL-6, IL-8, IL-12, IL-15, MCP-1, IP-10, Eotaxin, TGF-β1, TGF-β2, TGF-β3, MMP-1, MMP-3, MMP-9, COMP, and DcR3 or fragments thereof.

12. The method of any preceding method claim or the panel of any preceding panel claim, wherein the test biomarkers may comprise at least, or consist of, TNF-α, IL-6, IL-8, IL-12, IL-15, MCP-1, IP-10, Eotaxin, TGF-β1, TGF-β2, TGF-β3, MMP-1, MMP-3, MMP 9, COMP, DcR3, TIMP-1, ADAMTS-4, PIIANP or fragments thereof.

13. The method of any preceding method claim or the panel of any preceding panel claim, wherein the test biomarkers may comprise at least, or consist of IL-6, MCP-1, IP-10, TGF-β3, ADAMTS-4, TIMP-1, COMP and PIIANP or fragments thereof.

14. The method of any preceding method claim or the panel of any preceding panel claim, wherein the test biomarkers comprises one or more of IL-1β, TNF-α, IL-6, IL-8, IL-2, IL-12, IL-15, GM-CSF, IL-1Ra, IL-4, IL-10, IL-2R, RANTES, MIP-1α, MIP-1β, MCP-1, IP-10, Eotaxin, MIG TGF-β1, TGF-β2, TGF-β3, BMP-2, BMP-7 MMP-1, MMP-3, MMP-9, MMP-13, TIMP-1, ADAMTS-4 COMP, PIIANP, LIGHT, or DcR3.

15. The method of any preceding claim, wherein the step of comparing the statistical score with reference statistical scores generated from at least three reference group expression profiles further comprises:
querying a database of reference expression levels of the test biomarkers from a plurality of reference samples, wherein the database of known expression levels comprises the expression levels of at least the test biomarkers for samples of bodily fluids taken from subjects diagnosed with one or more of inflammatory arthritis, injury and osteoarthritis, in particular end stage osteoarthritis.

16. The method of any preceding method claim, wherein the at least one reference group expression profile is obtained by:
analyzing reference samples obtained from patients with a known diagnosis of either injury, inflammation or end stage osteoarthritis;
measuring the reference samples for the reference expression levels of at least the test biomarkers;
undertaking statistical analysis of the reference expression levels of each reference sample relative to the reference expression levels of the ensemble of reference samples to determine a relative expression profile for each reference sample; and
generating a reference group expression profile by mapping the relative expression profile of each reference sample to the known diagnosis of each reference sample.

17. The method according to any preceding method claim, wherein the step of calculating a statistical score further comprises:
undertaking statistical analysis of the expression levels of the test sample relative to the expression levels of the ensemble of reference samples to determine a relative test expression profile for the test sample; and
determining the statistical fit between the relative test expression profile and the relative expression profile and assigning a statistical score based on the statistical fit.

18. The method of any preceding method claim, wherein the statistical analysis uses partial least squares fit techniques and optionally or preferably wherein partial least squares discriminant analysis is used.

19. The panel of any preceding panel claim, wherein the expression levels of the test biomarkers are used to perform the method of any preceding method claim.

20. A system for calculating the probability that a subject has osteoarthritis, inflammatory arthritis or an injury, the system comprising: a test sample of bodily fluid obtained from a subject; a panel of test biomarkers; a processor for undertaking statistical analysis on the expression levels of the biomarkers from the panel; a database containing one or more reference group expression profiles; and a output device for signalling the results of the statistical analysis, wherein the processor determines a statistical score based on a comparison between the expression levels of the panel of test biomarkers in the test sample and the reference group expression profiles representing the probability that the expression levels of the test biomarkers in the test sample diagnose osteoarthritis, inflammatory arthritis or an injury, and in particular end stage osteoarthritis.

21. A system according to claim 20, wherein the system is used to assist in the diagnosis, prediction, monitoring or determining of end stage osteoarthritis and/or wherein the system also forms part of a test to assist diagnosis and monitor disease progression

22. A system for undertaking the method of any preceding method claim, the system comprising:
a panel according to any preceding panel claim;
a database containing one or more reference group expression profiles; and
an output device for displaying the statistical score.

23. A method according to any preceding method claim, further including the steps of:
obtaining sequential test samples over time; and
analysing the levels and pattern of test biomarker expression to determine change in disease status over said time.

24. A method according to any preceding method claim, further including the steps of: analyzing samples taken from a subject at various time points following initial diagnosis; and monitoring the changes in the biomarker panel expression profile to monitor osteoarthritis or joint injury progression and/or to monitor the efficacy of treatments/preventative regimes administered to a subject.

25. A method according to any preceding method claim, wherein the method further comprises the step of: determining the appropriate treatment of the subject.

26. Use of a panel of test biomarkers according to any preceding panel claim in the method of any preceding method claim to determine the osteoarthritis, inflammatory arthritis or joint injury status of a subject.

27. The use of a panel according to claim 26, wherein the determination of the expression profile of a biomarker panel of the invention in a synovial fluid sample for identifying the osteoarthritis, inflammatory arthritis or knee injury status of a subject, is in particular for identifying end stage osteoarthritis in a subjects knee.

28. A method of choosing the most appropriate treatment for a subject with joint injury or pain, the method including the steps of: performing the method of any preceding method claim on a sample, preferably a knee synovial fluid sample, from a subject and administering treatment based on the observed levels/profile of test biomarkers in the sample.

29. A diagnostic reagent for osteoarthritis comprising antibodies or synthetic antibodies (aptamers) for test biomarkers in a biomarker panel according to any preceding panel claim.

30. A kit comprising the diagnostic reagent of claim 29

31. A kit according to claim 30, wherein the antibodies are on a chip for high throughput screening.

32. A kit according to claim 30 or claim 31, wherein the kit comprises: a multi-well plate or microfluidic card or multi-plex chip prepared with reagents to capture and quantify the markers constituting the biomarker panel; a database containing disease reference profiles; and a computer module facilitating comparison of the test results with the reference panel using appropriate statistics.

33. A kit according to any one of claims 30 to 32 further comprising instructions for suitable operational parameters in the form of a label or separate insert.

34. A method, panel, kit or system according to any preceding claim, wherein the level of the three or more test biomarkers present in the sample is the concentration of the biomarker protein in the sample.

35. A method, panel, kit or system according to claim 34, wherein the level is determined by a suitable assay, such, as the use of any of the group comprising immunoassays, mass spectrometry, western blot, ELISA, immunoprecipitation, slot or dot blot assay, isoelectric focussing, SDS-PAGE and antibody microarray immunohistological staining, radio immuno assay (RIA), fluoroimmunoassay, an immunoassay using an avidin-biotin or streptoavidin-biotin system, quantitative PCR etc and combinations thereof.

36. A method, panel, kit or system according to claim 34 or claim 35 wherein the level is determined using targeted tandem mass spectrometry (MS) methods such as accurate inclusion mass spectrometry (AIMS), and quantitative selection reaction monitoring (Q-SRM).

37. A method according to any preceding method claim, wherein the method is carried out in-vitro.

38. A method, panel, kit or system according to any preceding claim, wherein the subject is a mammal.

39. A method according to claim 38, wherein the mammal is a human or a monkey, ape, cat, dog, cow, horse, rabbit or rodent.
